(19) United States
(12) Patent Application Publication
(10) Pub. No.: US 2012/0188511 A1
(43) Pub. Date: Jul. 26, 2012

(54) METHOD AND ANALYSIS SYSTEM FOR MEASURING THE GEOMETRY OF THE EYE

(75) Inventors: Gert Köst, Hannover (DE); Andreas Steinmüller, Wettenberg (DE)

(73) Assignee: Oculus Optikgerate GmbH, Wetzlar (DE)

(21) Appl. No.: 13/300,060

(22) Filed: Nov. 18, 2011

Related U.S. Application Data
(63) Continuation-in-part of application No. PCT/EP2010/056737, filed on May 17, 2010.

(30) Foreign Application Priority Data
May 18, 2009 (DE) 10 2009 021 770.3

(51) Int. Cl. A61B 3/10

(52) U.S. Cl. 351/211; 351/246; 351/205

(57) ABSTRACT

The invention relates to an ophthalmic analysis system and a method for measuring a geometry of an eye to be examined using the ophthalmic analysis system that comprises a first interferometric analysis system and a second non-interferometric analysis system. The first analysis system is used for obtaining measured data on optical boundary surfaces of the eye located on a measurement axis, and measured data describing relative distances, and the second analysis system is used for obtaining at least one set of image data on optical boundary surfaces located on the measurement axis. A processing device of the ophthalmic analysis system processes the measured data and the set of image data and corrects the set of image data using the measured data.

This is a Continuation-in-Part Application in the United States of International Patent Application No. PCT/EP2010/056737 filed May 17, 2010, which claims priority from German Patent Application No. 10 2009 021 770.3 filed May 18, 2009. The entire disclosures of the above patent applications are hereby incorporated by reference.

### FIELD OF THE INVENTION

The invention relates to a method and an ophthalmological analysis system for measuring the geometry of an eye to be examined, using a first interferometric analysis system and a second non-interferometric analysis system, wherein measured data describing relative distances of optical boundary surfaces of the eye on a measurement axis are obtained using the first analysis system, wherein at least one image data set is obtained from optical boundary surfaces on the measurement axis using the second analysis system, and a processing device of the ophthalmological analysis system processes the measured data and the image data set.

### BACKGROUND OF THE INVENTION

Methods for measuring the geometry of the eye and/or ophthalmological analysis systems that combine two different analysis systems, are sufficiently well known. For example, an ophthalmological analysis system that is formed by an interferometer and an imaging analysis system, is known from the state of the art. The imaging analysis system is used essentially to determine a position of an optical boundary surface and/or cornea in relation to the interferometer. The interferometer serves to determine the distance of the retina in relation to the interferometer so that an axis length of an eye can be determined from the two measured values.

Essentially non-interferometric imaging analysis systems have the disadvantage that non-interferometric imaging analysis systems are comparatively inaccurate in comparison with an interferometer. However, the optical boundary surfaces of the eye, for example, the outer and the inner surfaces of the cornea as well as a front and rear sides of the lens, can be determined by using imaging analysis systems and can be represented in a sectional view. The relative distances of the optical boundary surfaces from one another can be determined from an image data set obtained in this way.

Acquisition of measured values using an interferometer is comparatively more time-consuming because the interferometer must scan a measurement zone having a measurement point situated in it, for example, an optical boundary surface. This may occur by displacing a mirror and/or changing a length of an optical reference segment of the interferometer, for example. If the scanning process is performed for a long measurement segment comprising multiple optical boundary surfaces, then a comparatively long period of time is required. Only the distances of the optical boundary surfaces along one measurement axis can be determined with such a measurement, and it is impossible to determine a partial detail of an optical boundary surface such as a radius of curvature of a cornea, as is possible with various imaging analysis methods.

### SUMMARY OF THE INVENTION

The object of the present invention is therefore to propose a method for measuring the geometry of the eye and/or an ophthalmological analysis system that makes it possible to obtain the image information that describes the geometry of the eye with an improved accuracy.

In accordance with a first embodiment of the method, a method for measuring the geometry of the eye on an eye (15) that is to be examined using an ophthalmological analysis system (10), comprising a first interferometric analysis system (11) and a second non-interferometric analysis system (12), wherein measured data describing relative distances area obtained from optical boundary surfaces (26, 27, 29, 30, 32) of the eye on a measurement axis (16, 41) using the first analysis system, wherein at least one image data set is obtained from optical boundary surfaces on the measurement axis using the second analysis system, wherein a processing device (13) of the ophthalmological analysis system processes the measured data and the image data set, characterized in that the image data set is corrected by the processing device using the measured data.

In accordance with a second embodiment of the present invention, the first embodiment is further modified so that the second analysis system (12) is formed from a projection unit (34, 35) and an observation device (36, 37), wherein areas of the eye (15) defined with the projection unit are illuminated, and an image data set of the illuminated area is obtained by using the observation device. In accordance with a third embodiment of the present invention, the second embodiment is modified so that a Scheimpflug system having the projection device (34, 35) and the observation device (36, 37), which are arranged according to the Scheimpflug rule in relation to one another is used as the second analysis system (12). As is known to those of skill in the art, the Scheimpflug rule is a geometric rule that describes the orientation of the plane of focus of an optical system when the lens plane is not parallel to the image plane, as described by British Patent BP1196, incorporated herein by reference. See also, the Scheimpflug rule as described by U.S. Pat. No. 5,512,965 and U.S. Pat. No. 4,090,775, also incorporated herein by reference.

In accordance with a fourth embodiment of the present invention, the first embodiment, the second embodiment and the third embodiment are further modified so that the measurement axis (16, 41) is arranged to run along a projection plane (17) of the second analysis system (12). In accordance with a fifth embodiment of the present invention, the first embodiment, the second embodiment, the third embodiment and the fourth embodiment are further modified so that the correction of the image data set is performed after a comparison of a relative distance of at least two optical boundary surfaces (26, 27, 29, 30, 32) of the image data set with a relative distance of the same optical boundary surfaces of the measured data. In accordance with a sixth embodiment of the present invention, the first embodiment, the second embodiment, the third embodiment, the fourth embodiment and the fifth embodiment are further modified so that one dimension of the image data set is defined by the measurement axis (16, 41) and the image data set is corrected in this dimension.

In accordance with a seventh embodiment, the first embodiment, the second embodiment, the third embodiment, the fourth embodiment, the fifth embodiment and the sixth embodiment are further modified so that a plurality of image data sets is obtained in sequential order. In accordance with an eighth embodiment, the seventh embodiment is further modified so that a projection plane (17) of the second analysis system (12) is pivoted about an optical axis (14) of the eye (15). In accordance with a ninth embodiment, the first embodiment, the second embodiment, the third embodiment, the fourth embodiment, the fifth embodiment, the sixth embodiment, the seventh embodiment and the eighth embodiment are further modified so that a calibration of the second analysis system (12) is performed by means of the first analysis system (11).

In accordance with a tenth embodiment, the first embodiment, the second embodiment, the third embodiment, the fourth embodiment, the fifth embodiment, the sixth embodiment, the seventh embodiment, the eighth embodiment and the ninth embodiment are further modified so that the second analysis system (12) generates a first image data set, wherein a relative position of at least one optical boundary surface (26, 27, 29, 30, 32) is determined as a reference point for the first analysis system (11) on the basis of the image data set. In accordance with an eleventh embodiment, the ninth embodiment is further modified so that the measured data and the image data set are detected at the same time. In accordance with a twelfth embodiment, the first embodiment, the second embodiment, the third embodiment, the fourth embodiment, the fifth embodiment, the sixth embodiment, the seventh embodiment, the eighth embodiment, the ninth embodiment, the tenth embodiment and the eleventh embodiment are further modified so that the first analysis system (11) and the second analysis system (12) each emit electromagnetic radiation of different wavelength ranges. In accordance with a thirteenth embodiment, the first embodiment, the second embodiment, the third embodiment, the fourth embodiment, the fifth embodiment, the sixth embodiment, the seventh embodiment, the eighth embodiment, the ninth embodiment, the tenth embodiment, the eleventh embodiment and the twelfth embodiment are further modified so that a Michelson interferometer is used as the first analysis system (11).

In accordance with a fourteenth embodiment, an ophthalmological analysis system (10) for measuring the geometry of the eye on an eye (15) to be examined using a first interferometric analysis system (11) and a second non-interferometric analysis system (12), wherein measured data describing relative distances from optical boundary surfaces (26, 27, 29, 30, 32) on a measurement axis (16, 41) of the eye can be obtained using the first analysis system, wherein at least one image data set of optical boundary surfaces on the measurement axis can be obtained with the second analysis system, wherein the ophthalmological analysis system comprises a processing device (13) for processing the measured data and the image data set, characterized in that the processing device is designed so that the image data set can be corrected with the measured data. In accordance with a fifteenth embodiment, the fourteenth embodiment is further modified so that the first analysis system (11) and the second analysis system (12) are arranged in a shared housing.

The inventive method for measuring the geometry of an eye that is to be examined is performed using an ophthalmological analysis system, comprising a first interferometric analysis system and a second non-interferometric analysis system, wherein measured data describing relative distances from optical boundary surfaces of the eye situated on a measurement axis is obtained using the first analysis system, wherein at least one image data set is obtained from optical boundary surfaces on the measurement axis using the second analysis system, and a processing device of the ophthalmological analysis system processes the measured data and the image data set, and the image data set is corrected by the processing device using the measured data.

By correcting the image data set on the basis of the measured data of the interferometer, an improved accuracy corresponding essentially to the accuracy of the interferometer can be achieved for the image data set and thus for a geometric image representation and analysis. The processing device comprises suitable means for processing the measured data and the image data set as well as for graphical presentation of results. The processing device recognizes the optical boundary surfaces from the measured data and the image data set and corrects the image data set at least partially for the area of the image data set overlapping with the measured data. This measurement method may be performed in sections or for an entire axis length of the eye, wherein fundamentally all the interferometric and non-interferometric analysis systems that are known from the state of the art and are suitable, may be used for this measurement method.

The second analysis system may be formed by a projection unit and an observation device, such than defined areas of the eye are illuminated with the projection unit, and an image data set of the illuminated region can be obtained with the observation device. Such a structure of a second analysis system permits a simple means of obtaining an image data set of optical boundary surfaces of the eye.

Thus a Scheimpflug system with the projection device and the observation device arranged in relation to one another according to the Scheimpflug rule may be used as the second analysis system. The projection device may include slit-lamp lighting of the eye along the optical axis, wherein then a camera positioned according to the Scheimpflug principle can detect the cross-sectional area of the eye illuminated in this way. The resulting longitudinal sectional image of the eye may then preferably show the optical boundary surfaces of the cornea and the lens. From the resulting image data set, the processing device can easily calculate the relative distances of the optical boundary surfaces.

A particularly accurate correction of the image data set is possible if the measurement axis is arranged so that it runs along a projection plane of the second analysis system. A direct comparison of the relative distances of the optical boundary surfaces determined with the first and second analysis systems is possible through the course of the measurement axis through the projection plane. The measurement axis may preferably correspond to the optical axis of the eye. Alternatively, however, it is also possible to arrange the measurement axis at a distance from the optical axis in an eccentric and/or peripheral area of an anterior chamber of the eye, depending on the position of the projection plane in relation to the optical axis. This may be advantageous in particular when especially precise measured values are to be determined in this area.

In a simple variant of the process, the image data set can be corrected by the processing device after comparing a relative distance of at least two optical boundary surfaces of the image data set with a relative distance of same optical boundary surfaces of the measured data. Since the relative distance resulting from the image data set is comparatively inaccurate, the relative distance contained in the measured data may serve as a reference measure according to which the image data set is corrected and/or modified. An especially accurate image data set can be obtained when the measured data set contains more than two optical boundary surfaces, for example, all the optical boundary surfaces contained in the image data set, or two optical boundary surfaces and/or their positions that are an especially great distance apart from one another, and the processing device uses same for the correction.

The image data set may be corrected in a particularly simple manner if one dimension of the image data set is defined by the measurement axis and the image data set is corrected in this dimension. The image data set may thus be compressed or stretched easily in one direction of the measurement axis depending on the required correction, until a position of the optical boundary surfaces contained in the image data set coincides with a position of same in the boundary surfaces contained in the measured data. For the case when more than two optical boundary surfaces are used for a correction, it is possible to correct the compression or stretching of the image data set on the basis of a non-linear function.

A plurality of image data sets may also be obtained in a sequential order. For example, the second analysis system may obtain a series of parallel sectional images of equal distances apart along a line running across the optical axis. The processing device can then combine the individual image data sets of these sectional images to form an image data set that allows a three-dimensional representation of the eye. In this context, it may be advantageous if the first analysis system performs a measurement of at least one optical boundary surface on recording a sectional image and/or an image data set. This facilitates the combining of the corresponding image data sets with respect to a reference point obtained in this way. Possible movements of the eye occurring during the sequential detection of the image data sets may also be corrected much more accurately.

In another alternative of generating a sequential image data set, a projection plane of the second analysis system may be pivoted about an optical axis of the eye. The projection plane may thus be rotated about the optical axis of the eye. It is advantageously possible to obtain a particularly high data density within an area around the optical axis. With respect to the alternatives described above for obtaining measured data, in this case the measurement axis may correspond to the optical axis, so that only a single measurement is necessary with the first analysis system. If the measurement axis does not correspond to the optical axis, the measurement axis may be positioned on a diameter of a circle coaxially with the optical axis, following the respective projection planes. This may then yield a three-dimensional image that has been improved even further with geometric dimensions of the eye.

It is also possible by means of the first analysis system to perform a calibration of the second analysis system. In the simplest variant, a single correction of a first image data set may be performed by means of the measured data, wherein all possible additional image data sets may also be corrected and/or adapted on the basis of the correction values determined once.

It is particularly advantageous when the second analysis system generates a first image data set, wherein a relative position of at least one optical boundary surface is determined as a reference point for the first analysis system on the basis of this image data set. The first analysis system may then scan the optical boundary surfaces known from the first image data set in a targeted manner, in such a way that long scanning distances are avoided. The detection of the required measured data can be greatly accelerated by the restricted examination range of the first analysis system.

Further time optimization of the method is made possible if the measured data and the image data set are detected at the same time. Then the image data set can be corrected immediately after receiving the data.

The first analysis system and the second analysis system may each emit electromagnetic radiation with wavelength ranges different from one another. The electromagnetic radiation may preferably be in the visible or infrared wavelength range. Superimposing the wavelength ranges of the first and second analysis systems that has a negative effect on data extraction, can thus be avoided advantageously.

A Michelson interferometer may be used for the first analysis system. Such an interferometer has proven to be especially suitable for use with this method.

The inventive ophthalmological analysis system for measuring the geometry of the eye to be examined thus comprises a first interferometric analysis system and a second non-interferometric analysis system, wherein measured data describing relative distances of optical boundary surfaces of the eye situated on a measurement axis can be obtained with the first analysis system, wherein at least one image data set can be obtained from optical boundary surfaces on the measurement axis by using the second analysis system, wherein the ophthalmological analysis system comprises a processing device for processing the measured data and the image data set and the processing device is designed so that the image data set can be corrected with the measured data.

It is especially advantageous if the first and the second analysis systems are arranged in a shared housing. A number of components can be eliminated by using the components jointly. For example, only one each of the housing itself, the necessary add-on parts, a power supply unit and some optical and electronic modules are needed.

Additional advantageous embodiments of the ophthalmological analysis system are derived from the description of the features of those embodiments that refer back to the method embodiment of the first embodiment of the current invention.

### BRIEF DESCRIPTION OF THE DRAWINGS

The present invention is explained in greater detail below with reference to the accompanying drawings:

FIG. 1 shows a schematic diagram of the structure of an ophthalmological analysis system;

FIG. 2 shows a front view of the eye to be analyzed;

FIG. 3 shows a sectional view of the eye to be analyzed.

### DETAILED DESCRIPTION OF THE INVENTION

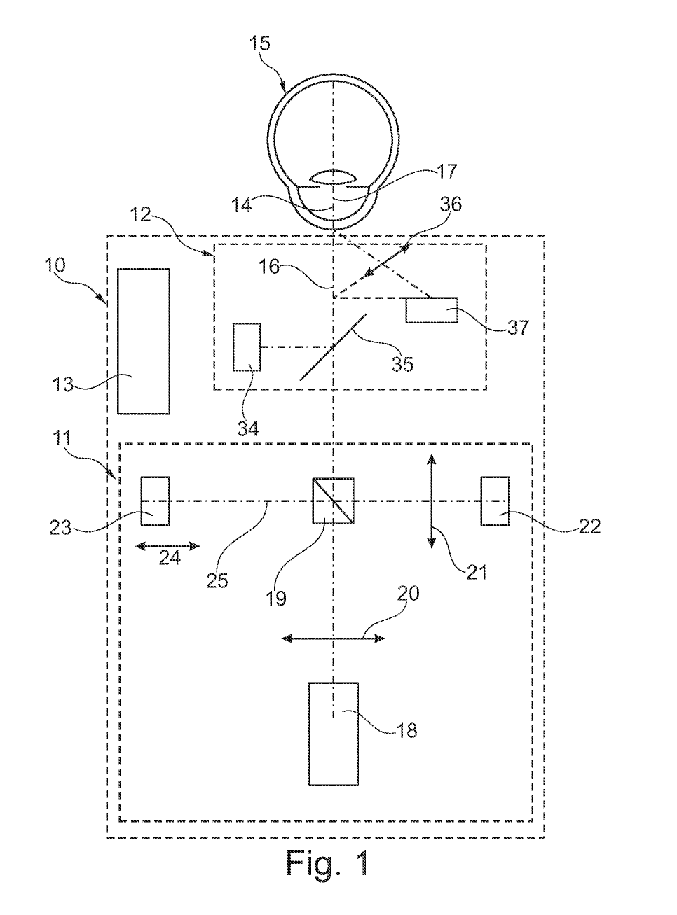

FIG. 1 shows a schematic diagram of the structure of an ophthalmological analysis system 10, comprising a first interferometric analysis system, designed as an interferometer 11, and a second non-interferometric analysis system, designed as a Scheimpflug recording system 12, as well as a processing device 13. The ophthalmological analysis system 10 is arranged in relation to an optical axis 14 of an eye 15 to be examined, wherein the optical axis 14 corresponds to and/or passes through a measurement axis 16 of the interferometer 11 and a projection plane 17 of the Scheimpflug recording system 12. The interferometer 11 is formed essentially by a reading light source 18, an optical beamsplitter 19, lens configurations 20 and 21, a detector device 22 and a mirror device 23. In particular the mirror device 23 is arranged to be longitudinally displaceable along the double arrow 24 so that the length of a reference segment 25 is variable. By a shift in the mirror device 23, various regions of the eye 15 which are located on the optical axis 14 may be scanned. No further explanation of a known function of the interferometer 11 will be given here. In particular measured data describing the relative positions of optical boundary surfaces on the axis 14, such as a front surface 26 of a cornea 28, a posterior surface 27 of the cornea 28, an anterior surface 29 of a lens 31, a posterior boundary surface 30 of the lens 31 and an area 32 of the retina 33 according to the diagram in FIG. 3 may be obtained.

The Scheimpflug recording system 12 comprises a split-lamp lighting device 34, a partially transparent mirror 35, a lens configuration 36 and a camera device 37. A light gap, which is not described in further detail here, is projected into the eye 15 by means of the mirror 35 in agreement with the optical axis 14 and/or the measured axis 16, so that its transparent constituents can be visualized through light scattering in the projection plane 17 within the eye 15. According to the Scheimpflug rule, the lens configuration 36 and the camera device 37 are arranged in relation to the plane of the projection 17 so that an image of the projection plane 17, which is not shown here, can be detected as a sharp image and converted to an image data set by the camera device 37.

The measured data of the areas 26, 27 and 29, 30 as well as 32, obtained using the interferometer 11, are sent to the processing device 13, so that these relative distances of the areas 26, 27 and 29, 30 as well as 32 are calculated based on the measurement axis 16 and/or the optical axis 14. The image data set obtained using the Scheimpflug recording system 12 is also sent to the processing device 13 that calculates the position of the areas 26, 27 and 29, 30 and optionally 32 by image processing in relation to the measurement axis 16 and/or optical axis 14. The relative distances calculated on the basis of the measured data are comparatively more accurate than the relative distances calculated on the basis of the image data set, so the image data set is corrected by the processing device 13, so that the relative distances of the image data set correspond to the relative distances of the measured data. It is provided here in particular that the image data set is modified in the direction of the measurement axis 16 and/or optical axis 14, so that the image can be compressed and/or stretched. The correction described above may also be performed alone on the basis of a single relative distance.

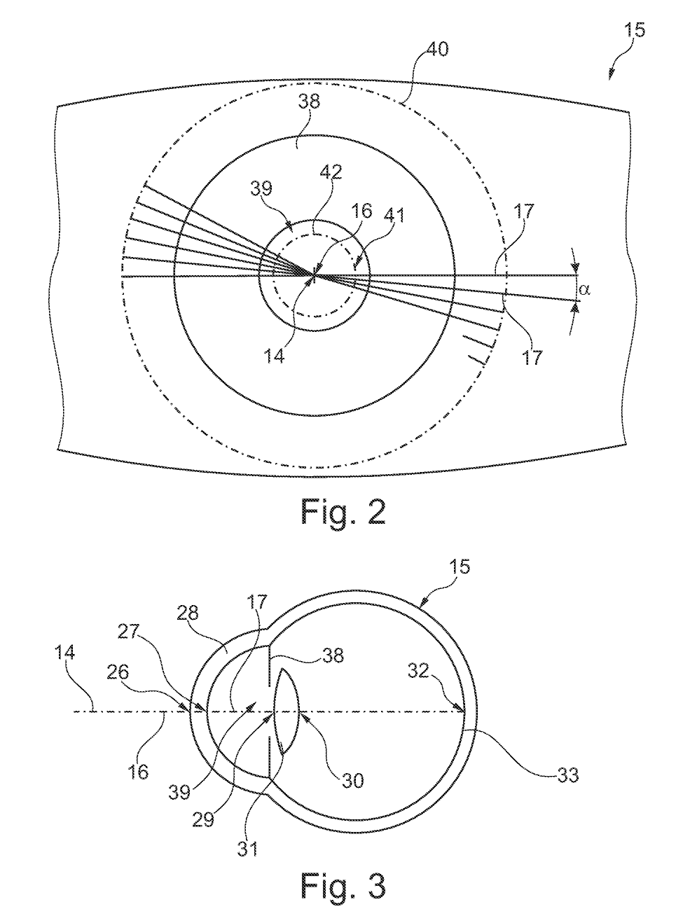

FIG. 2 shows a sectional front view of the eye 15, with the iris 38 with a pupil aperture 39 discernible within the eye. In addition, the projection plane 17 is represented as a horizontal line through which the optical axis 14 runs. The Scheimpflug recording system 12 is designed so that, after obtaining a first image data set, the projection plane 17 can be pivoted by an angle α about the optical axis 14 to obtain a second image data set and n additional image data sets in sequential order. The image data sets are obtained within a circle diameter 40 described by the projection plane 17. The measurement axis 16 falls in the optical axis 14 as described above, so that all the image data sets obtained can be corrected with a single set of measured data. Alternatively, FIG. 2 shows a measurement axis 41, which is pivoted with the projection plane 17 by the angle α about the optical axis 14 on a circle diameter 42. This embodiment requires an optical analysis system (not shown here) with a corresponding configuration of an interferometer and/or beam deflection of the measurement axis 41. The measured data thereby obtained for each image data set makes it possible to derive a corrected three-dimensional representation and/or geometric determination of the topography of the eye in the area scanned by the interferometer about the optical axis 14.

1. A method for measuring the geometry of the eye on an eye that is to be examined using an ophthalmological analysis system, wherein the ophthalmological analysis system comprises a first interferometric analysis system and a second non-interferometric analysis system, the method comprising the steps of:
(a) using the first analysis system to obtain, from optical boundary surfaces of the eye on a measurement axis, measured data describing a relative distances area;
(b) using the second analysis system to obtain at least one first image data set from the optical boundary surfaces on the measurement axis; and
(c) processing the measured data and the first image set using a processing device of the ophthalmological analysis system, wherein
the first image data set is corrected by the processing device using the measured data.

2. The method according to claim 1, comprising the additional step of:
(d) obtaining second image data using the second analysis system, wherein the second analysis system is formed from a projection unit and an observation device, wherein areas of the eye defined with the projection unit are illuminated, and the second image data set obtained using the observation device is of the illuminated area.

3. The method according to claim 2, comprising the additional step of:
(e) arranging a Scheimpflug system containing the projection device and the observation device according to the Sheimpflug rule.

4. The method according to claim 1, comprising the additional step of:
(d) arranging the measurement axis to run along a projection plane of the second analysis system.

5. The method according to claim 1, comprising the additional step of:
(d) correcting the first image data set after comparing a relative distance of at least two optical boundary surfaces of the first image data set with a relative distance of the same optical boundary surfaces of the measured data.

6. The method according to claim 1, comprising the additional step of:
(d) defining one dimension of the first image data set by the measurement axis and correcting the first image data set in this dimension.

7. The method according to claim 1, comprising the additional step of:
(d) obtaining a plurality of additional image data sets in sequential order.

8. The method according to claim 7, comprising the additional step of:
(e) pivoting a projection plane of the second analysis system about an optical axis of the eye.

9. The method according to claim 1, comprising the additional step of:
(d) performing a calibration of the second analysis system by means of the first analysis system.

10. The method according to claim 1, comprising the additional step of:
(d) generating a second image data set, wherein a relative position of at least one optical boundary surface is determined as a reference point for the first analysis system on the basis of the first image data set using the second analysis system.

11. The method according to claim 9, comprising the additional step of;
(e) detecting the measured data and the first image data set at the same time.

12. The method according to claim 1, comprising the additional step of:
(d) emitting electromagnetic radiation of different wavelength ranges using the first analysis system and the second analysis system.

13. The method according to claim 1, wherein the first analysis system is
a Michelson interferometer.

14. An ophthalmological analysis system for measuring the geometry of the eye on an eye to be examined, the ophthalmological analysis system comprising:
(a) a first interferometric analysis system; that obtains measured data describing relative distances from optical boundary surfaces on a measurement axis of the eye;
(b) a second non-interferometric analysis system, that obtains at least one image data set of optical boundary surfaces on the measurement axis; and
(c) an ophthalmological analysis system comprising a processing device for processing the measured data and the image data set, wherein
the processing device is configured so that the image data set is correctable using the measured data.

15. An analysis system according to claim 14,
wherein the first analysis system and the second analysis system are arranged in a shared housing.

16. The method according to claim 9, comprising the additional step of:
(e) generating a second image data set, wherein a relative position of at least one optical boundary surface is determined as a reference point for the first analysis system on the basis of the first image data set using the second analysis system.

17. The method according to claim 3, comprising the additional step of:
(f) arranging the measurement axis to run along a projection plane of the second analysis system.

18. The method according to claim 4, comprising the additional step of:
(g) correcting the first image data set after comparing a relative distance of at least two optical boundary surfaces of the first image data set with a relative distance of the same optical boundary surfaces of the measured data.

19. The method according to claim 11, comprising the additional step of:
(f) emitting electromagnetic radiation of different wavelength ranges using the first analysis system and the second analysis system.

20. The method according to claim 19, wherein the first analysis system is a Michelson interferometer.
